(19) United States
(12) Patent Application Publication
(10) Pub. No.: US 2006/0058801 A1
(43) Pub. Date: Mar. 16, 2006

(54) Locking screw

(76) Inventors: Andre Schlienger, Munchenstein (CH); Peter Senn, Waldenburg (CH); Markus Buettler, Mumliswil (CH)

Correspondence Address: JONES DAY, 222 EAST 41ST STREET, NEW YORK, NY, 10017-6702, US

(21) Appl. No.: 11/237,595

(22) Filed: Sep. 27, 2005

Related U.S. Application Data
(63) Continuation of application No. PCT/CH03/00202, filed on Mar. 28, 2003.

(51) Int. Cl. A61B 17/58

(52) U.S. Cl. 606/73

(57) ABSTRACT

A locking screw comprising a screw head, and a screw shaft having an external thread, and a diameter d. One end of the screw shaft is connected to the screw head, and the other end of the screw shaft being a free end. A center line, defined as the line connecting the centers of gravity of the axially sequential, orthogonal cross-sectional surfaces of the locking screw, has a first end point at the screw head and a second end point at a free end of the screw shaft, is not a continuously straight line, and the center line coincides with a point of inflection. Due to the configuration of the locking screw, the clearance between the transverse borehole of the modullary pin and of the locking screw (1) can be eliminated.

### CROSS REFERENCE TO RELATED APPLICATION

This application is a continuation of International Patent Application No. PCT/CH2003/000202, filed Mar. 28, 2003, the entire contents of which is expressly incorporated herein by reference.

### TECHNICAL FIELD

The invention relates generally to locking screws.

### BACKGROUND OF THE INVENTION

Locking of medullary pins is known in the art. Locking screws used for locking medullary pins may be introduced into the transverse boreholes of the medullary pin either with the help of an imaging method (X-ray control) or a targeting device. In both cases, a certain amount of inaccuracy in targeting the locking pin may be unavoidable. That is, the tip of a locking screw may not be aligned exactly coaxially with the middle axis of the transverse borehole and, instead, deviates therefrom by a certain amount. So that the locking screw can pass through the transverse borehole in spite of this targeting error, the external diameter of the screw is underdimensioned (smaller) relative to the diameter of the transverse borehole. If the targeting accuracy remains in the range of this underdimensioning, the locking screw can be passed through the transverse borehole without a problem in spite of the targeting error.

Because of the underdimensioning, a certain clearance results between the locking screw and the transverse borehole. This clearance defines the amount by which the main fragments of the bone, which are fixed in the corresponding locking hole by means of locking screws, can move relative to the pin and, accordingly, because of the rigidity of the pin, also relative to other main bone fragments fastened with the same pin. Together with the flexibility of the material and of the overall device, this may prevent successful healing or delay healing significantly. The clearance between the locking screw and transverse borehole may be unavoidable so as to guarantee the applicability of the locking for the surgeon. However, this clearance is undesirable in certain situations, such as metaphysical fragments.

Even pins with a full cross section, which may have an internal thread in the locking hole, may have a clearance. The internal thread merely prevents the pin from moving axially on the locking screw.

### SUMMARY OF THE INVENTION

The present invention is to provide a remedy for the above-discussed disadvantages. An object of the present invention is to create a locking screw, with which the clearance, existing between it and the transverse borehole in a locking medullary pin, can be eliminated.

The present invention accomplishes the objective set out above with a locking screw comprising a screw head, and a screw shaft having an external thread, and a diameter d. One end of the screw shaft is connected to the screw head, and the other end of the screw shaft being a free end. A center line, defined as the line connecting the centers of gravity of the axially sequential, orthogonal cross-sectional surfaces of the locking screw, has a first end point at the screw head and a second end point at a free end of the screw shaft, is not a continuously straight line, and the center line coincides with a point of inflection.

A method of installing at least one locking screw into a medullary pin includes the steps of inserting the locking screw into the borehole of the medullary pin 10, where the transverse borehole has a cross-sectional profile with a maximum extent “a” measured in the direction of center line and a maximum extent “b” measured perpendicularly to “a”, such that a>b and a>d<b, and turning the locking screw in a standard fashion through the transverse borehole, where relatively thin and soft corticalis yields as the locking screw is screwed through it, so that there is no strain over the thickness of the corticalis. In the region of the medullary pin 10, the locking screw is stretched because of a reaction of the wall of the transverse borehole and an increased force is required for screwing in the locking screw, resulting in a higher holding force results

An advantage achieved by the present invention is due to the inventive locking screw clearance between the transverse boreholes of the medullary pin and the locking screw can be eliminated. Further advantages of the present invention are that the accuracy of introducing the pin and the time required by the surgeon remain within the previous limits, the firmness of the locking screw is retained, and the extraction in the event of a possible screw breakage is assured.

Other objectives and advantages, in addition to those discussed above, will become apparent to those skilled in the art during the course of the description of the embodiments of the invention which follows. In the description, reference is made to accompanying drawings, which form a part thereof, and which illustrate examples of the invention. Such examples, however, are not exhaustive of the various embodiments of the invention, and therefore, reference is made to the claims that follow the description for determining the scope of the invention.

### BRIEF DESCRIPTION OF THE DRAWINGS

FIG. 1 shows a perspective view of a locking screw,

FIG. 2 shows a longitudinal section through the locking screw of FIG. 1,

FIG. 3 shows a longitudinal section through a locking medullary pin with a transverse borehole, into which the locking screw of FIG. 1 is introduced, and

FIG. 4 shows a longitudinal section of another embodiment of the locking screw.

### DESCRIPTION OF THE PREFERRED EMBODIMENTS

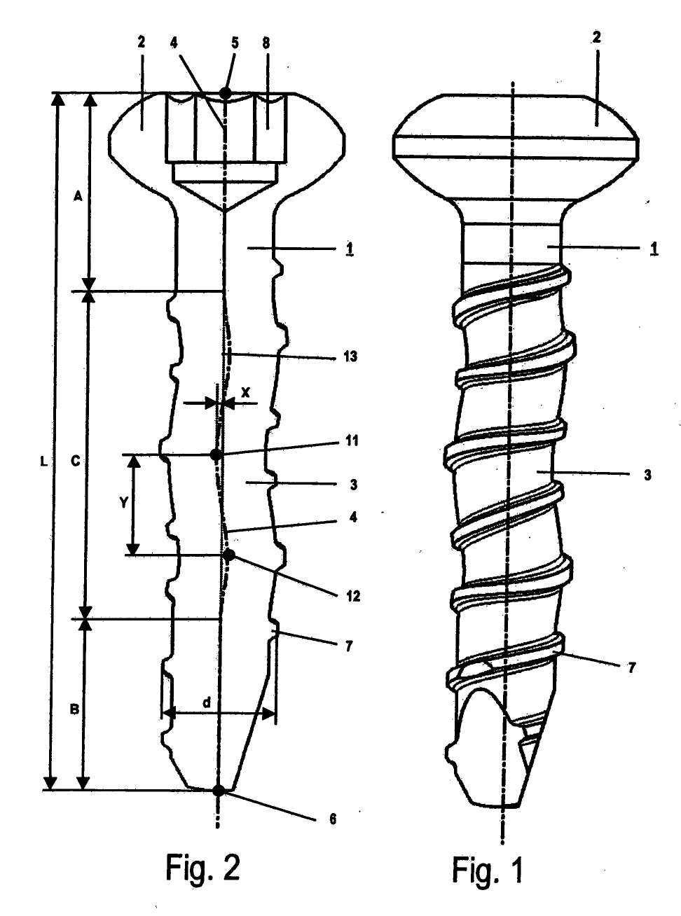

The locking screw, shown in FIGS. 1 and 2, comprises a screw head 2 with a hexagonal socket 8, a screw shaft 3 with an external thread 7. A center line 4, being a line connecting the centers of gravity of the axially sequential orthogonal cross-sectional surfaces of the locking screw, has a first end point 5 at the screw head 2 and a second end point 6 at the free end of the screw shaft 3. Unlike straight screws, the center line 4 of the present invention is not a straight line and, instead, as shown in FIG. 2 (in the length region C defined below), consists of a coiled line, which lies in the plane of the drawing and has two points of inflection 11, 12. A connecting straight line 13 extends through the two end points 5, 6 of the center line 4, and deviates in places from the center line 4 by the variable amount “x”. In the example shown, the maximum deviation of “x”, measured at the points of inflection of the center line 4, is 0.2 mm.

The center line 4 is divided into sections A, B and C between the first and second end points 5, 6, which are at a distance L from one another. Section A extends from the first end point 5 at the screw head 2 by the amount of 0.10 L to 0.25 L towards the second end point 6 at the free end of the screw shaft 3. Section B extends from the second end point 6 at the free end of the screw shaft 3 by the amount of 0.10 to 0.25 L towards the first end point 5 at the screw head 3. Section C is disposed between the two sections A and B, and has a length C=(L−A−B), where the center line 4 in sections A and B being essentially linear and extending coaxially to one another.

In the case of a another embodiment, Section A extends from the first end point 5 at the screw head 2 by the amount of ⅙ L in the direction of the second end point 6 at the free end of the screw shaft 3 and extends essentially in a straight line. Section B extends from the second end point 6 at the free end of the screw shaft 3 by the amount of ⅙ L in the direction of the first end point 5 at the screw head 2 and also extends essentially in a straight line, coaxially with section A. Section C is disposed between the two sections A and B and has a curvature, as described above.

An advantage of these embodiments of the locking screw 1 is that locking in the opposite corticalis is accomplished by a rotational movement about the connecting straight line and the locking in the corticalis at the screw head 2 essentially is along the borehole axis.

Another embodiment of the locking screw 1 is shown in FIG. 4, in which the center line 4 consists of three mutually offset straight lines. The distance “x” between the center line 4 and the connecting straight line 13 in section C between the two end points 5, 6 in this embodiment is 0.15 mm.

In the case of another embodiment, a connecting straight line 13, extending between the two end points 5, 6 of the center line 4, has a distance “x” from the center line 4 at least at one place between the two end points, where “x” is greater than 0.01 mm and preferably greater than 0.10 mm. The distance “x” preferably observes the condition 0.01 d<x<0.30 d and more preferably the condition 0.05 d<x<0.20 d, where “d” is the diameter of the diameter of the screw shaft 3 in mm.

In the case of a further embodiment, the distance “x” observes the condition 0.05(b−d)<x<0.35(b−d) and more preferably the condition 1.5(b−d)<x<2.2(b−d), where “d” is diameter of the screw shaft 3 in mm and “b” is the diameter of the transverse borehole 9 in mm (discussed later).

In a further embodiment, the center line 4 is curved S-shaped or eccentric in section C only.

The center line 4 may have a point of inflection, preferably only in section C. It may also have at least two points of inflection at a distance “y” from one another, also preferably only in section C. The distance between two adjacent points of inflection, “y”, substantially observes the condition D=ny, where “n” is an odd number and “D” is the diameter of the medullary pin 10.

Furthermore, the center line 4 may also lie in one or more planes.

In a further embodiment, the center line 4 is formed by several straight lines, which are transposed relative to one another, so that a simpler manufacturing process results.

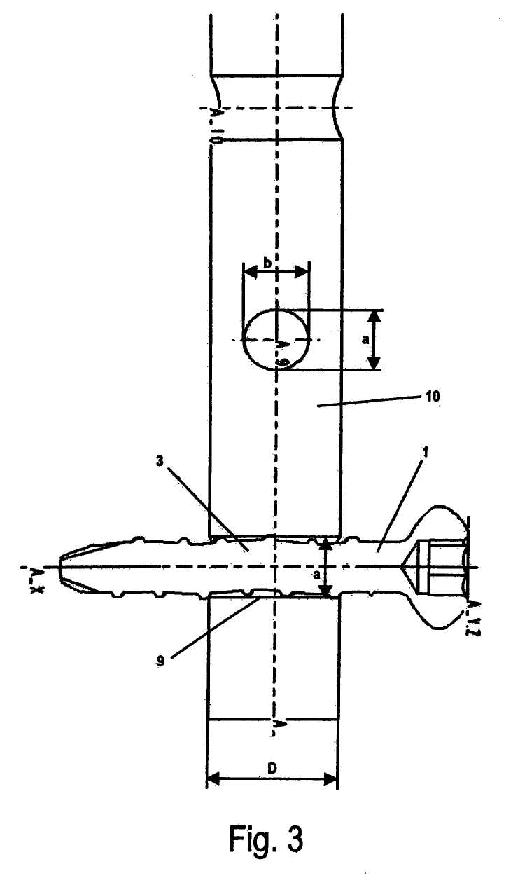

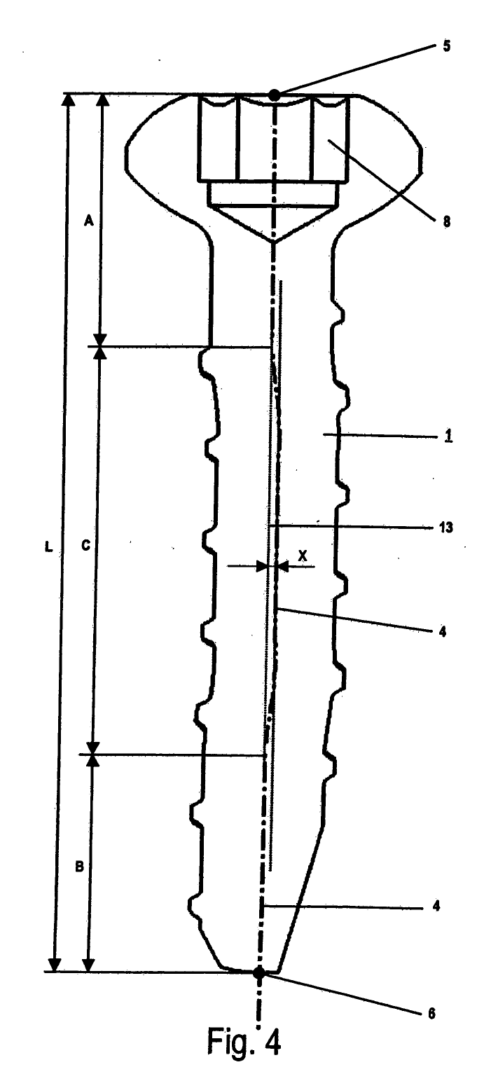

The locking screw 1 may be used together with a locking medullary pin 10, which has at least one transverse borehole 9. FIG. 3 shows how the locking screw 1 is introduced into the transverse borehole 9 of a medullary pin 10. Preferably, the transverse borehole 9 has a cross-sectional profile with a maximum extent “a”, measured in the direction of the center line 4, and a maximum extent “b”, measured perpendicularly to “a”. Diameter “d” of screw shaft 3 (FIG. 2) may be smaller than the dimension “a” of the transverse borehole 9, such that in one embodiment of the present invention the following conditions may exist a>b and a>d<b.

The cross-sectional profile may also be circular with a=b. Preferably, the condition 0.70 b<d<0.95 b, and more preferably 0.8 b<d<0.9 b, applies. Distance “x” preferably observes the condition x<(b−d+1 mm), where “b” is the diameter of the transverse borehole 9 in mm and “d” is the diameter of the screw shaft 3 in mm.

In the case of another embodiment of the invention, the locking screw 1 may not have any rotational axis of symmetry.

A brief description of a surgical procedure of screwing the locking screw 1 into the transverse borehole 9 of a medullary pin 10 follows in order to explain the invention further.

A surgeon inserts the locking screw 1 into the borehole 9 of the medullary pin 10 and turns the locking screw 1 in a standard fashion through the transverse borehole 9. The relatively thin and soft corticalis yields as the locking screw 1 is screwed through it, so that there is no strain over the thickness of the corticalis. In the region of the medullary pin 10, the locking screw 1 is stretched somewhat because of the reaction of the wall of the transverse borehold 9, so that an increased force is required for screwing in the locking screw 1 and a higher holding force results. In the event of a cannulation of the medullary pin 10, the locking screw 1 winds through the entry opening of the transverse borehole 9 of the medullary pin 10 into the transverse borehole 9, since the diameter D of the medullary pin 10 is larger than the distance y between the two points of inflection 11, 12. Due to the screwing-in movement or due to the drilling force of the surgeon, an elastic deformation is forced upon the locking screw 1, no later than when it takes hold of the opposite corticalis. This leads to an angularly stable locking of the medullary pin 10.

1. A locking screw comprising:
a screw head;
a screw shaft having an external thread, and a diameter d,
wherein one end of the screw shaft is connected to the screw head, and the other end of the screw shaft being a free end,
wherein a center line, defined as the line connecting the centers of gravity of the axially sequential, orthogonal cross-sectional surfaces of the locking screw, having a first end point at the screw head and a second end point at a free end of the screw shaft, is not a continuously straight line, and
wherein the center line coincides with a point of inflection.

2. A locking screw according to claim 1, wherein the locking screw does not have a rotational axis of symmetry.

3. A locking screw according to claim 1, wherein the center line lies in a plane.

4. A locking screw according to claim 1, wherein the center line lies in more than one plane.

5. A locking screw according to claim 1, wherein the center line is formed from several mutually transposed straight lines.

6. A locking screw according to claim 1, wherein a straight connecting line passing through the two end points of the center line is at a distance x from the center line at least at one point between the two end points, and
wherein x>0.01 mm and more preferably x>0.10 mm.

7. A locking screw according claim 6, wherein the distance x fulfills the condition 0.01 d<x<0.30 d and more preferably the condition 0.05 d<x<0.20 d, where d is the diameter of the screw shaft.

8. A locking screw according to claim 1,
wherein the length of the center line between the two end points is L,
wherein the center line is divided between the two end points into three sections A, B and C, such that section A extends a distance between 0.10 L and 0.25 L from the first end point towards the second end point, section B extends a distance between 0.10 to 0.25 L from the second end point towards the first end point, and section C, being disposed between the two sections A and B, having a length C=(L−A−B), and
wherein the center line in sections A and B being essentially linear and extending coaxially to one another.

9. A locking screw according to claim 8, wherein the center line is curved S-shaped or is eccentric only in section C.

10. A locking screw according to claim 8, wherein the center line has a point of inflection preferably only in section C.

11. A locking screw according to claim 8, wherein center line has at least two points of inflection preferably only in section C at a distance y from one another.

12. A locking screw according to claim 1, wherein the diameter “d” of the screw shaft is substantially constant.

13. A method of installing at least one locking screw into a medullary pin, said method comprising the steps of:
inserting the locking screw into the borehole of the medullary pin 10, wherein the transverse borehole has a cross-sectional profile with a maximum extent “a” measured in the direction of center line and a maximum extent “b” measured perpendicularly to “a”, such that a>b and a>d<b; and
turning the locking screw in a standard fashion through the transverse borehole, wherein relatively thin and soft corticalis yields as the locking screw is screwed through it, so that there is no strain over the thickness of the corticalis,
wherein in the region of the medullary pin 10, the locking screw is stretched because of a reaction of the wall of the transverse borehole and an increased force is required for screwing in the locking screw, resulting in a higher holding force results.

14. A method according to claim 13, wherein the cross-sectional profile is circular with a=b and a condition of 0.70 b<d<0.95 b and more preferably a condition of 0.8 b<d<0.9 b applies.

15. A method according to claim 13, wherein a distance “x” fulfills a condition of x<(b−d+1 mm), where “b” is the diameter of the transverse borehole in mm, “d” is the diameter of the screw shaft in mm, and “x” is a distance between a connecting straight line and a center line.

16. A method according to claim 15, wherein the distance “x” fulfills a condition of 0.05 (b−d)<x<0.35 (b−d) and more preferably a condition of 1.5 (b−d)<x<2.2 (b−d), where “d” is the diameter of the screw shaft in mm.

17. A method according to claim 13, a distance “y” between two adjacent points of inflection substantially fulfills a condition of D=ny, where “n” is an odd number and “D” the diameter of the medullary pin.
